(19) United States
(12) Patent Application Publication
(10) Pub. No.: US 2005/0288589 A1
(43) Pub. Date: Dec. 29, 2005

(54) Surface model parametric ultrasound imaging

(75) Inventors: Helene C. Houle, Sunnyvale, CA (US); Christian W. Galuschky, Munich (DE); Marcus Schreckenberg, Freising (DE); John I. Jackson, Menlo Park, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignees: Siemens Medical Solutions USA, Inc.; Tom Tec Imaging Systems GmbH

(21) Appl. No.: 11/158,582

(22) Filed: Jun. 21, 2005

Related U.S. Application Data
(60) Provisional application No. 60/583,280, filed on Jun. 25, 2004.

(51) Int. Cl. A61B 8/02

(52) U.S. Cl. 600/450

(57) ABSTRACT

Parametric imaging of a surface is provided on a medical diagnostic ultrasound imaging system. A bull's eye or Beutel surface representing the scanned tissue, such as a portion of the heart, is formed from planar views, such as apical 4 chamber, apical 2 chamber and long axis views of the heart. Dynamic clips or videos of the parametric imaging provide temporally useful information to a user. The parametric imaging may include information determined from data at different locations or different times, such as strain, velocity, tissue displacement, velocity, or wall thickness. The ultrasound data may be responsive to contrast agents. The ultrasound data may be acquired with a three-dimensional scan.

### RELATED APPLICATIONS

The present patent document claims the benefit of the filing date under 35 U.S.C. § 119(e) of Provisional U.S. patent application Ser. No. 60/583,280, filed Jun. 25, 2004, which is hereby incorporated by reference.

### BACKGROUND

This present description relates to medical imaging. In particular, parametric imaging for strain, strain rate or other motion parameters using surface models is provided.

Ultrasound is used to assist in diagnosis of heart conditions. Doppler velocity and/or B-mode imaging of the heart provides off-line or real-time images of the heart. Two or three dimensional images are viewed as static images or dynamic clips. However, other analysis or characteristics may be derived from ultrasound information, such as strain or strain rate.

In “Strain And Strain Rate Parametric Imaging. A New Method For Post Processing Three Standard Apical Planes To 3-/4-Dimensional Images. Preliminary Data On Feasibility, Artefact And Regional Dyssynergy Visualization,” Støylen et al. describe off-line visualization for heart diagnosis. However, the methods and systems described have some undesired limitation.

### BRIEF SUMMARY

By way of introduction, the preferred embodiments described below include methods, systems and computer readable media for parametric imaging of a heart with ultrasound. The parametric imaging capability is located on a medical diagnostic ultrasound imaging system. Dynamic clips or videos of the parametric imaging provide temporally useful information to a user. The parametric imaging may include values determined from data at different locations or different times, such as strain or tissue tracking values. The parametric values may be derived from heart cycle phase information. The ultrasound data may be responsive to contrast agents. The ultrasound data may be acquired with a three-dimensional scan. Any one or combination of features disclosed herein may be used.

In a first aspect, a method is provided for parametric imaging of a heart with ultrasound. Ultrasound data representing the heart along at least two different planes is acquired. The ultrasound data represents the heart at different times. A motion parameter is determined as a function of the ultrasound data at different locations, different times or both the different locations and different times. A dynamic, parametric surface is displayed as a function of the motion parameter.

In a second aspect, a system is provided for parametric imaging of a heart with ultrasound. A beamformer is operable to form ultrasound data representing the heart along at least two different planes as a function of scanning. A processor connects with the beamformer within the system. The processor is operable to determine a motion parameter as a function of the ultrasound data at different locations, different times or both the different locations and different times. A display is operable to display a parametric surface as a function of the motion parameter.

In a third aspect, a method is provided for parametric imaging of a heart with ultrasound. Ultrasound data representing the heart along at least two different planes is acquired. The ultrasound data represents the heart at different times. A curved line is identified in each of the planes. A motion parameter is determined as a function of the ultrasound data on the curved lines. A dynamic, parametric surface is displayed as a function of the motion parameter and the curved lines.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a diagnostic medical ultrasound imaging system for parametric imaging;

FIG. 2 is a flow chart of one embodiment of a method for parametric imaging of a surface;

FIG. 3 is a graphical representation of a two dimensional scan of a heart;

FIG. 4 is a graphical representation of a M-mode display in one embodiment;

FIG. 5 is a graphical representation of one embodiment of a three dimensional representation of a surface; and

FIG. 6 is a graphical representation of one embodiment of derivation of and the resulting polar plot parametric surface.

### DETAILED DESCRIPTION OF THE DRAWINGS AND PRESENTLY PREFERRED EMBODIMENTS

In one embodiment, parametric imaging for strain and strain rate use surface models. Visualization of cardiac function is achieved by displaying a dynamic parametric image on an ultrasound system. The dynamic parametric image has colors that represent some aspect of the motion (i.e., strain rate or strain) of the myocardium (cardiac muscle). The colors change as a function of time, showing how these motion parameters change with time. The colors are shown on a moving surface, or shell. The moving surface represents the relative shape of the myocardium in general, or its endocardial surface in particular, throughout the cardiac cycle. The colors are additionally or alternatively shown on a polar plot (i.e., a bulls-eye representation) where different regions within the disk correspond to standard regions within the heart's left ventricle. For some displays, a static (e.g. end-diastolic) surface model is provided. Other combinations of one or more of these features and other features herein may be used, such as for imaging different portions of the heart or different organs with the same or different motion parameters.

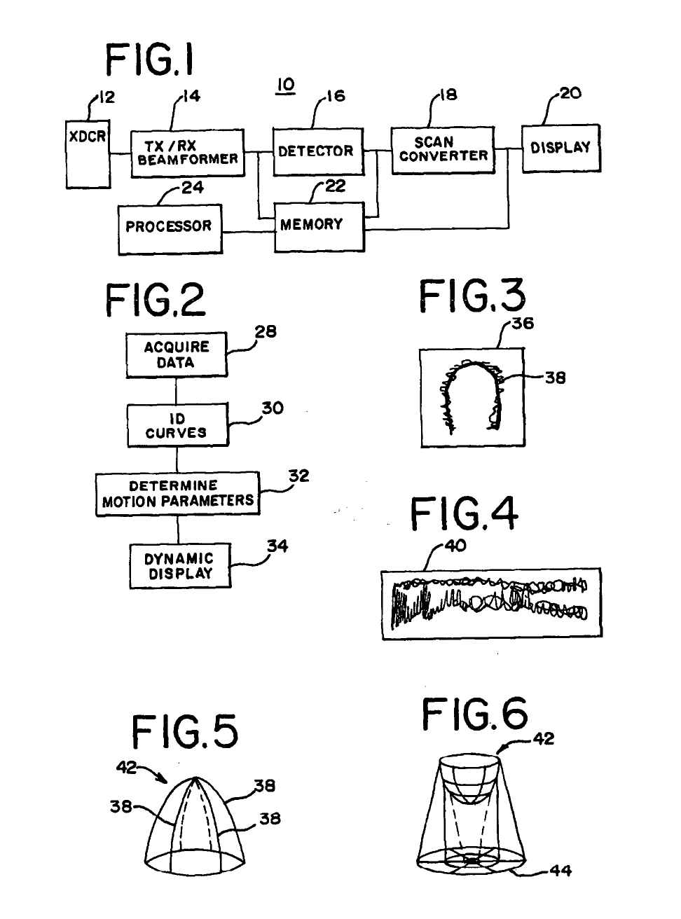

FIG. 1 shows a system 10 for parametric imaging of a heart with ultrasound. The system 10 includes a transducer 12, a beamformer 14, a detector 16, a scan converter 18, a display 20, a memory 22, and a processor 24. Additional, different, or fewer components may be provided. For example, the processor 24 and memory 22 are provided on a separate system, such as a remote workstation or computer. The system 10 is a medical diagnostic ultrasound imaging system, such as a cart or portable system for real-time scanning of a patient. Post processes in an “off-line” mode may be provided on the system, allowing analysis of ultrasound data without transfer to remote systems.

The transducer 12 is a one, 1.25, 1.5, 1.75, 2 or other multi-dimensional probe. The transducer 12 is permanently or releasably connected with the system 10. Handheld, wobble, catheter, endocavity, transesophageal (TEE) or other transducers 12 are used. In one embodiment, the transducer 12 is a single array, but multiple arrays of elements may be provided. The transducer 12 includes or does not include an absolute position sensor or other device for determining a current position or displacement associated with the transducer 12.

The beamformer 14 is a transmit, receive or both transmit and receive beamformer. As a transmit beamformer 14, a plurality of waveform generators or pulsers, delays, phase rotators, amplifiers, filters and/or other structures are provided in channels for generating relatively delayed and apodized electrical waveforms for the elements of the transmit aperture on the transducer 12. As a receive beamformer 14, a plurality of amplifiers, filters, delays, phase rotators, summers and/or other structures are provided in channels for summing relatively delayed and apodized receive signals. A single summer may alternatively be provided. The beamformer 14 includes a transmit and receive switch for selecting between transmit and receive paths or operation.

The beamformer 14 is operable to form ultrasound data along at least two different planes as a function of scanning. For example, at least two different planes are scanned by moving the transducer 12 to a new orientation or changing a scanning parameter to obtain a different plane with the transducer 12 in a same orientation or position. By applying different delay and apodization profiles, acoustic energy is generated to scan along different scan lines. Echo signals are delayed, apodized, and summed to form ultrasound data representing tissue, fluid or other structure along the scan lines. A complete scan of a region generates a frame of data for a given time. For flow or Doppler processing, the frame of data for a given time may be associated with multiple transmissions. Given the speed of sound in tissue, the region is scanned at a substantially same time for a frame of data. More rapid scanning is provided in alternative embodiments by multiple beam transmission or reception or by plane wave transmission. By repeating the scan at different times, multiple frames of data for a same region at different times are provided.

Different planes are scanned by repositioning the transducer 12, a transmit aperture, a receive aperture or a scan plane position. Multiple planes are also scanned by electronically and/or mechanically scanning a volume, such as scanning with a wobbler array.

Any organ or tissue may be imaged. In one embodiment, different planes representing the heart are scanned. For example, the user manually positions the transducer 12 to acquire ultrasound data from apical 4 chamber, apical 2 chamber and apical long axis scans of the heart. Additional, fewer or different standard or non-standard views may be used. Each of the scans is repeated at different times throughout at least a portion of a heart cycle. Scanning throughout one or more heart cycles may be used to increase an amount of data acquired for analysis. In one embodiment, data for a same view or scan plane and different heart cycles is combined temporally to provide ultrasound data representing a single heart cycle. The ultrasound data for one or more cycles or portions of cycles is time warped to temporally align the data for combination. Alternatively, the acquisition of ultrasound data over multiple heartbeats is used to show changes from one heartbeat to the next or for over-sampling and averaging to reduce artifact and noise within the data sets. The heart cycle timing relative to acquisition is derived from the ultrasound data or obtained from an ECG monitor input.

In one embodiment, a position sensor records the relative position of the acquisition planes. To know the position of a contour, plane or tissue, freehand acquisition uses position sensors. Using a rotational device, the image acquisition may be automated. The user positions the transducer 12 on a defined view (reference view) and the system 10 then acquires other views automatically. Scanning a volume electronically alternatively provides position information. The position information is later used for relative alignment of data from the different planes. Alternatively, an alignment is assumed, such as where standard views are used. In yet another embodiment, a TEE transducer 12 with a fixed rotational axis is used to acquire the ultrasound data. The later derived surface may be based on the endocardial surface or on the endocardial contours from multiple 2D planes.

The detector 16 is an intensity (e.g., B-mode or M-mode), velocity (e.g., Doppler velocity), Doppler tissue velocity, contrast agent (e.g., phase inversion), harmonic (e.g., receiving at a second harmonic of a transmitted frequency), or other now known or later developed detector or combinations thereof. In one embodiment, the detector outputs velocity estimates for each spatial location or a subset of spatial locations within a scanned plane. In other embodiments, an intensity is output for each spatial location or for a selected line or curved line within a scanned plane. Similarly, contrast agent data based on Doppler or intensity processes may be output. The ultrasound data input is detected by the detector 16. The detector 16 outputs detected ultrasound data to the scan converter 18. For integrated versions and/or for offline solutions, the processor 24 may directly work on the ultrasound data prior to scan conversion.

Velocity estimates are angle corrected. For angle correction, scans of a same plane or spatial location from two transducer positions or two aperture positions are used to determine a true in-plane velocity vector. Alternatively, the system 10 estimates or the user inputs a flow direction for angle correction. True longitudinal and transversal strain or strain rate components are computed from angle corrected velocities. Angle dependency is corrected for all or most points in regions of interest in the scan planes, such as along contours corresponding to the heart wall or muscle. Alternatively, velocities along the scan lines without angle correction are used.

The scan converter 18 converts the ultrasound data from a polar coordinate or acquisition coordinate format to a Cartesian or display coordinate format. The scan converted ultrasound data is provided to the display 20. Any types of images may be displayed, such as B-mode, M-mode, Velocity, or combinations thereof. The display 20 also displays the parametric surface images generated from the ultrasound data. The display 20 is a CRT, LCD, projector, plasma screen, touch screen or other now known or later developed display device.

The memory 22 is a CINE memory, RAM, hard disc, CD, DVD, removable media, cache, buffer, system memory or other now known or later developed memory for storing one or more frames of ultrasound data. In one embodiment, the memory 22 stores clips or a plurality of frames of data for each of the different scanned planes. The memory 22 acquires the ultrasound data from one or more different locations along the ultrasound data path between the beamformer 14 and the display 20.

The processor 24 is a control processor, central processing unit, general processor, application specific integrated circuit, field programmable gate array, digital signal processor, graphics processing unit, analog circuit, digital circuit, combinations thereof or other now known or later developed device for determining motion parameter values and/or generating a display surface. The processor 24 connects directly or indirectly with the beamformer 14 within the system 10. For example, the beamformer 14 or other portions of the ultrasound data path are within a same housing of a medical diagnostic ultrasound imaging system 10.

The processor 24 is operable to determine a motion parameter as a function of the ultrasound data. Motion parameters include displacement, strain, strain rate, torsion, velocity, change in wall thickness or combinations thereof. The motion parameters are determined from ultrasound data at different locations, different times or both the different locations and different times. For example, strain or strain rate is determined from velocity ultrasound data representing different spatial locations in a same frame of data. As another example, displacement is determined by correlation of intensity speckle or tissue between frames of data acquired at different times. A Fourier analysis may be used to determine displacement. As yet another example, the motion parameter represents the relative phasing as compared to the heart cycle. For example, the phase or amplitude parameter disclosed in U.S. Pat. Nos. ______ and ______ (application Ser. Nos. 10/713,453 and ______ (attorney reference no. 2004P01562US01), the disclosures of which are incorporated herein by reference, is used. In another example, velocity is determined using Doppler techniques, analysis of b-mode data, or combinations thereof, such as described in U.S. Pat. No. 6,527,717, the disclosure of which is incorporated herein by reference.

The values for the motion parameter are determined for one or a plurality of different spatial locations. For example, the motion parameter is determined from the ultrasound data associated with heart tissue from each of the views, such as the apical four chamber (A4C), apical two chamber (A2C) and apical long axis (ALA) views. For each view, the motion parameters are determined for each spatial location or for spatial locations of interest. For example, the beamformer 14 acquires the ultrasound data as color M-mode data associated with curved lines corresponding to the heart tissue for each of the scans. The ultrasound data is formatted as a frame of data for a two dimensional region or as a set of velocities along the curved line as a function of time (color M-mode). The ultrasound data for velocity information is acquired only along the curved lines or within regions including the curved lines. The curved lines or regions of interest are identified automatically or manually. By scanning the different views of the heart, two dimensional (2D) coordinates and velocity samples of a contour 38 (see FIG. 3) representing a curved M-Mode 40 (see FIG. 4) positioned on the myocardium are acquired. The processor 24 determines the motion parameters for the spatial locations along the curved lines 38.

The processor 24 generates the motion parameter values for the same or corresponding spatial locations at different times. Ultrasound data representing the planes or views at different times during the heart cycle are processed. Values for the motion parameters are calculated for each of the different times. The curved lines or region of interest are tracked through multiple images. The tracking occurs automatically, such as using thresholds, speckle or tissue tracking or automated border detection. Alternatively, the user manually indicates a position of the region of interest or curved line for each frame of data. Based on the regions of interest or curved lines identified for different times, motion parameter values are determined for generating a parametric surface as a video clip running through at least a portion of a heart cycle. Alternatively, the processor 24 generates a dynamic 3D surface model in some proprietary format and any type of display described herein is used. For example, a display format allows the view perspective to be chosen during review.

The processor 24, using a graphics card, the scan converter 18, a frame buffer, combination thereof or without other components, generates a parametric surface as a function of the motion parameter values. The display 20 is operable to receive and display the parametric surface. The parametric surface is a two or three dimensional representation of a three dimensional portion of the scanned tissue, such as the heart. The motion parameter values are mapped to the surface.

In one embodiment shown in FIG. 5, the parametric surface is a three dimensional representation 42. For example, the representation 42 is of a portion of the heart. The curved lines 38 (see FIG. 3) are positioned relative to each other. In FIG. 5, three such curved lines 38 from A4C, A2C and ALA views are shown with about 60 degree spacing between each curved line 38. Approximated, estimated or actual relative positioning may be used, such as 30, 30, 120 degree spacing. The shape or contours formed by the relative placement of the curved lines 38 generally represents a shape of the heart or other structure at a given time. The curved lines 38 are positioned based on expected or known relationship, such as through the use of the three standard views of the heart, based on position sensing of the transducer 12, or based on assumption. A Beutal display of the heart or a portion of the heart is formed.

Strain, strain rate, velocity, change in wall thickness, displacement or other motion parameter values are known for spatial positions along each of the curved lines 38. The motion parameter values are mapped to the surface 42. Parameters other than motion parameters, such as wall thickness at a given time, may alternatively or additionally be mapped to the surface. Static parameters may be mapped onto the Beutel (e.g., display a static Beutel with Echo Phase Imaging Information). Gray scale, color or both gray scale and color mapping are used. Texture mapping, look-up table or other mapping is used. The resolution of the mapping is binary or more complex. For example, strain is separated into two or more ranges. Each range is displayed with a different color or shade.

For spatial locations on the surface 42 for which data is not available or acquired, such as between the curved lines 38, the motion parameter values are interpolated. Spherical interpolation is used, but other interpolation or extrapolation may be used. Applying a heart model based on standard views, the three dimensional (3D) surface 42 is reconstructed using interpolation. Data at a similar longitude, latitude, nearest neighbors or other motion parameter values are selected for weighted interpolation to a given spatial location on the three dimensional surface defined by the curved lines 38. Interpolation generates motion parameter values for some or most of the surface 42.

In another surface for display on the display 20, a polar plot 44 is generated as shown in FIG. 6. The contours or three dimensional shape 42 formed by the curved lines 42 is projected onto a two dimensional surface as the polar plot 44. FIG. 6 shows the projection of the portion of the heart where an apex is mapped to the center of the polar plot 44. In other embodiments, the three dimensional surface 42 is projected at other angles onto the polar plot 44. The motion parameter values or mapped display values (e.g., color or gray scale) are projected. Interpolation is performed prior to or after projection. The polar plot 44 provides a two dimensional parametric surface representing the structure of interest, such as the heart.

The parametric surface 42, 44 is used to assess tissue function, such as systolic and diastolic function of the heart. The three dimensional representation of the parametric surface 42 provides a 3D model of the heart which enables a global visualization of contraction and relaxation of the heart.

To assist in visualization, the parametric surface 42, 44 is displayed dynamically. The data for the M-mode image 40 or other data representing the curved lines 38 at different times is arranged in sequence. After interpolation for each given time within the sequence, the parametric surface 42, 44 is displayed dynamically. To simplify interpolation, a fixed relative transformation or relationship between the curved lines 38 over time is assumed. Alternatively, the position of the curved lines 38 relative to each other varies as a function of time. The interpolation accounts for the variation. For a parametric surface of the heart, cardiac function is visualized during a portion or an entire heart cycle. For example, the dynamic parametric surface 42, 44 has colors that represent some aspect of the motion (e.g., displacement, velocity, strain rate, strain, phase, or torsion) of the myocardium (cardiac muscle). The colors change as a function of time, showing how these motion parameters change with time.

The colors are shown on a moving surface, or shell. As the shape or other characteristic of the curved lines 38 changes, the shape of the surface 42, 44 changes. The moving surface represents the relative shape of the myocardium in general, or its endocardial surface in particular, throughout the cardiac cycle. In an alternative embodiment, the colors are shown on a static surface 42, 44 with only the colors or other display values changing as a function of time.

Additional indications may be added to the parametric surfaces 42, 44. For example, one type of motion parameter controls one characteristic of the display values (e.g., brightness or gray scale) and another type of motion parameter controls a different characteristic of the display values (e.g., color). As another example, landmarks, such as LVOT, MV, AV, ANTERIOR WALL, SEPTUM, and/or RV, for visualization help for better understanding of the orientation of a 3D surface model are added as annotations to the parametric surface 42, 44.

Where a user desires objective information associated with the parametric surface, specific values may be displayed. For example, a localized region on the parametric surface 42, 44 is identified automatically or by the user. In one embodiment, the user positions a curved line different than the one used to form the parametric surface. An M-mode image, waveform or quantitative values are derived and displayed from the data of the parametric surface 42, 44. For example, peak velocity, time to peak velocity, A-wave velocity, mean strain, maximum strain or other now known or later developed quantitative values are calculated.

FIG. 2 shows a method for parametric imaging of a heart with ultrasound. Additional, different or fewer acts may be provided, such as performing acts 28, 30 and 32 without act 34. The acts are performed in the order shown or a different order. The system 10 or a different system implements the acts.

In act 28, ultrasound data is acquired. The ultrasound data represents a desired tissue or structure, such as representing the heart. The ultrasound data corresponds to different positions within a volume, such as acquiring ultrasound data along at least two different planes. For the heart, the data is acquired from A4C, A2C and ALA views. FIG. 3 shows a two dimensional scan of one view of the heart. Other views may be used. The data is acquired by scanning along different planes or positions using two or three dimensional (volume) scans. Any now known or later developed type of data, such as intensity, Doppler tissue, velocity, contrast agent or combinations thereof may be used.

The ultrasound data is acquired at different times. Different planes are scanned at the same or different times. For each plane or region, multiple scans are performed, such scanning for at least a portion of or the entire heart cycle. FIG. 4 shows a curved M-mode scan 40 representing data acquired along the curved line 38 of FIG. 3 over multiple heart cycles. The acquired ultrasound data represents different spatial perspectives of the same region over a similar or same period of time. For example, scans of the heart in each of three different standard views are acquired at different times. Ultrasound data representing the corresponding region is acquired over a same portion or the entire heart cycle for each of the views.

In act 30, a curved line 38, such as within the planes 36 of the different 2D views, is identified for each of the scanned regions, such as within the planes of the different 2D views. In the example of FIG. 3, the curved line 36 identifies the ultrasound data associated with heart wall tissue. Curved lines 38 identifying related tissue are identified for the other views.

The curved lines 38 are identified manually through tracing or computer assisted manual tracing (i.e. identifying the curved line 38 after the user indicates the location of one or more landmarks). Alternatively, the curved lines 38 are identified automatically by applying an algorithm. The curved lines 38 are thin, such as one pixel wide, or thicker, such as 5 mm or other thickness wide.

The curved lines 38 are identified in one frame of data and then tracked to other frames of data. For example, velocity information is used to track movement of different portions of the curved line 38 throughout a sequence, such as disclosed in U.S. Pat. No. ______ (application Ser. No. 10/861,268). As another example, speckle or tissue tracking with correlation, minimum sum of absolute differences or other function tracks the position of the curved lines 38 through a sequence. Alternatively, manual tracing or automatic identification of each curved line 38 within a sequence is used independent of the curved lines 38 identified for other frames of data within the sequence.

By identifying the curved lines 38 throughout a sequence, ultrasound data associated with the tissue of interest, such as the heart wall, at different times is selected. For example, FIG. 4 shows a colored (Doppler tissue velocity) M-mode data corresponding to the curved line 38 tracking the heart wall throughout a sequence. Where the curved lines 38 have a thickness associated with multiple samples, the samples are averaged, selected or otherwise combined to provide data for each of a plurality of spatial locations along the curved line 38.

In act 32, motion parameters are determined as a function of the ultrasound data. The motion parameters are determined for a same type of motion, such as strain, strain rate, torsion, velocity, change in wall thickness, relative phase of a cycle or other motion parameters. The motion parameters are calculated from ultrasound data for different locations, the different times or both the different locations and different times. For example, strain and strain rate are calculated from ultrasound data representing different spatial locations in a same frame of data. Tissue displacement values are calculated from ultrasound at different times and spatial locations.

Values of the motion parameter are calculated for each of the curved lines 38. For example, the ultrasound data from the color M-mode image 40 of FIG. 4 is used to calculate the motion parameters for each spatial location along the sequence of curved lines 38. The motion parameter represents a motion characteristic of the heart tissue in this example. In particular, the motion parameter for the heart wall, such as strain or strain rate, is calculated for the heart wall locations throughout a sequence.

The motion parameters are mapped to display values. For example, motion parameters are mapped to color (e.g., RGB) or gray scale values. One or more maps or mapping functions may be available. Depending on the application or user selection, a map is selected for mapping the motion parameters or a sequence. The motion parameters modulate the display values. For example, the display value is selected as a function of the identification of relative phasing of motion of a spatial location on the heart wall relative to the heart cycle.

In act 34, a parametric surface is displayed as a function of the motion parameter. The display values are displayed on an image. The parametric surface represents the motion parameters as particular time. The parametric surface is also formed as a function of the curved lines 38. The assumed, set or tracked relative position of the curved lines is used to form the contour of the parametric surface. For example, FIG. 5 shows a three dimensional representation 42 of at least a portion of the heart corresponding to the curved lines 38. The relative position of the curved lines 38 from different views at a same or similar time provides the framework or contour of the image. Both the endocardial and epicardial surfaces may be tracked and used to display a common or adjacent parametric surfaces, showing relative twist, mass, shear strain or other characteristics. As another example, the relative position of the curved lines 38 defines the relative locations of data within a two dimensional polar plot 44 shown in FIG. 6.

For spatial locations on the three dimensional representation 42 or the polar plot 44 not at a curved line 38, data is interpolated from the motion parameters for the nearest curved lines. Display values may alternatively be interpolated. The closeness of the curved line provides a relative weighting of the contribution of data from different curved lines. Spherical or other interpolation is used. Alternatively, a nearest neighbor selection is used. The interpolated or other data for the parametric surface is or is not spatially filtered.

The three dimensional representation 42 or polar plot 44 is formed for each temporal position in the sequence, such as for each time sample of the color M-mode data. Any one or selected group of the parametric surfaces is displayed in response to user input. Each parametric surface represents one or more motion characteristics of the tissue of interest at a given time in the sequence. By displaying the sequence or a portion of the sequence without interruption or further user input, the parametric surface is dynamically displayed. For example, a video clip of the parametric surface representing a portion of the heart is displayed. By displaying the dynamic parametric surface in synchronization with the heart cycle, the user may more likely understand or be able to diagnose heart motion abnormalities or heart disease.

Instructions for implementing the methods are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, filmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer or system.

While the invention has been described above by reference to various embodiments, it should be understood that many changes and modifications can be made without departing from the scope of the invention. For example, a parameter based on spatial differences or relationships, such as wall thickness, is used for the display as an alternative or in addition to a motion parameter. It is therefore intended that the foregoing detailed description be regarded as illustrative rather than limiting, and that it be understood that it is the following claims, including all equivalents, that are intended to define the spirit and scope of this invention.

1. A method for parametric imaging of a heart with ultrasound, the method comprising:
acquiring ultrasound data representing the heart along at least two different planes, the ultrasound data representing the heart at different times;
determining a parameter value as a function of the ultrasound data at different locations, the different times or both the different locations and different times; and
displaying a dynamic, parametric surface as a function of the motion parameter value.

2. The method of claim 1 wherein displaying comprises displaying the parametric surface as a video clip running through at least a portion of a heart cycle.

3. The method of claim 2 wherein displaying comprises displaying in synchronization with the heart cycle.

4. The method of claim 1 wherein acquiring the ultrasound data comprises:
acquiring the ultrasound data from apical four chamber, apical two chamber and apical long axis views at different times in at least a portion of the heart cycle; and
identifying the ultrasound data associated with heart tissue from each of the views;
wherein determining the parameter value comprises determining the parameter value from the ultrasound data associated with the heart tissue.

5. The method of claim 4 wherein acquiring the ultrasound data comprises acquiring color M-mode data associated with curved lines corresponding to the heart tissue for each of the views; and
wherein determining the parameter value comprises determining the parameter value for a plurality of spatial locations along the curved lines.

6. The method of claim 1 wherein determining the parameter value comprises determining a strain, a strain rate, velocity, wall thickness, tissue displacement or combinations thereof for a plurality of spatial locations of the heart.

7. The method of claim 1 wherein displaying comprises displaying a two dimensional polar plot projection of at least a portion of the heart.

8. The method of claim 1 wherein displaying comprises displaying a three dimensional representation of at least a portion of the heart.

9. The method of claim 5 wherein displaying comprises displaying a three dimensional representation of at least a portion of the heart, the three dimensional representation having contours derived as a function of the curved lines and the parametric surface interpolated between the curved lines on the three dimensional representation from the parameter values of the spatial locations along the curved lines.

10. The method of claim 1 wherein acquiring comprises acquiring the ultrasound data as a function of contrast agents, intensity or both contrast agents and intensity.

11. The method of claim 1 further comprising:
annotating the parametric surface.

12. The method of claim 1 wherein acquiring comprises acquiring as a function of a volume scan.

13. The method of claim 1 further comprising:
mapping the motion parameter value as a function of a selected color map.

14. The method of claim 1 wherein displaying the dynamic, parametric surface comprises forming the surface as a function of a spatial relationship between the at least two different planes.

15. The method of claim 1 wherein acquiring comprises acquiring over at least first and second heart cycles; and
further comprising:
temporally aligning data for the first heart cycle with data for the second heart cycle.

16. A system for parametric imaging of a heart with ultrasound, the system comprising:
a beamformer operable to form ultrasound data representing the heart along at least two different planes as a function of scanning;
a processor connected with the beamformer within the system, the processor operable to determine a parameter value as a function of the ultrasound data at different locations, different times or both the different locations and different times; and
a display operable to display a parametric surface as a function of the parameter value.

17. The system of claim 16 wherein the processor is operable to generate the parameter value for the parametric surface as a video clip running through at least a portion of a heart cycle.

18. The system of claim 16 wherein the beamformer is operable to form the ultrasound data from apical four chamber, apical two chamber and apical long axis scans at different times in at least a portion of a heart cycle; and
wherein the processor is operable to determine the parameter value from the ultrasound data associated with heart tissue from each of the views.

19. The system of claim 16 wherein the beamformer is operable to acquire the ultrasound data as color M-mode data associated with curved lines corresponding to the heart tissue for each of the scans; and
wherein the processor is operable to determine the parameter value for a plurality of spatial locations along the curved lines.

20. The system of claim 16 wherein the parameter value comprises a strain, a strain rate, velocity, wall thickness, tissue displacement or combinations thereof for a plurality of spatial locations of the heart.

21. The system of claim 16 wherein the display is operable to display a two dimensional polar plot projection of at least a portion of the heart.

22. The system of claim 16 wherein the display is operable to display a three dimensional representation of at least a portion of the heart.

23. The system of claim 19 wherein the display is operable to display a three dimensional representation of at least a portion of the heart, the three dimensional representation having contours derived as a function of the curved lines and the parametric surface interpolated between the curved lines on the three dimensional representation from the parameter values of the spatial locations along the curved lines.

24. The system of claim 16 wherein the beamformer and processor are within a same housing of a medical diagnostic ultrasound imaging system.

25. The system of claim 16 wherein the processor is operable to modulate a display value as a function of a phase of a heart cycle.

26. A method for parametric imaging of a heart with ultrasound, the method comprising:
acquiring ultrasound data representing the heart along at least two different planes, the ultrasound data representing the heart at different times;
identifying a curved line in each of the planes;
determining a motion parameter value as a function of the ultrasound data on the curved lines; and
displaying a dynamic, parametric surface as a function of the motion parameter value and the curved lines.

27. The method of claim 26 wherein acquiring comprises acquiring the ultrasound data from apical four chamber, apical two chamber and apical long axis views at different times in at least a portion of a heart cycle;
wherein identifying comprises identifying the ultrasound data associated with heart tissue from each of the views; and
wherein determining the motion parameter value comprises determining the motion parameter value from the ultrasound data associated with the heart tissue.

28. The method of claim 27 wherein acquiring the ultrasound data comprises acquiring color M-mode data associated with the curved lines corresponding to the heart tissue for each of the views; and
wherein determining the motion parameter value comprises determining the motion parameter value for a plurality of spatial locations along the curved lines.

29. The method of claim 26 wherein displaying comprises displaying a three dimensional representation of at least a portion of the heart, the three dimensional representation having contours derived as a function of the curved lines and the parametric surface interpolated between the curved lines on the three dimensional representation from the motion parameter values of the spatial locations along the curved lines.

30. The method of claim 26 wherein determining the motion parameter value comprises determining the motion parameter value as a function of ultrasound data at different locations, different times or both the different locations and different times.

31. The method of claim 1 wherein displaying the dynamic, parametric surface as the function of the motion parameter value comprises displaying dynamic parameter data on a surface with static geometry.
